(19) United States
(12) Patent Application Publication
(10) Pub. No.: US 2005/0079602 A1
(43) Pub. Date: Apr. 14, 2005

(54) Enclosure for processing biological samples having a removable framework element

(75) Inventors: Laurent Mimaud, Nantes (FR); Veronique Ollivier, Coueron (FR)

Correspondence Address: YOUNG & THOMPSON, 745 SOUTH 23RD STREET, 2ND FLOOR, ARLINGTON, VA, 22202, US

(73) Assignee: Jouan, Saint-Herblain (FR)

(21) Appl. No.: 10/958,543

(22) Filed: Oct. 6, 2004

(30) Foreign Application Priority Data
Oct. 8, 2003 (FR) 0311768

(51) Int. Cl. C12M 1/34

(52) U.S. Cl. 435/287.2; 435/289.1

(57) ABSTRACT

This enclosure comprises a body which itself comprises: a structure which internally delimits a processing space which opens at the outer side of the structure by way of an opening,
at least one framework element which is fixed in a removable manner to the structure in order to delimit, in the opening (13), at least two zones for access to separate regions of the processing space. At least two gates are mounted so as to be movable on the structure in order selectively to close and open the access zones.

Application, for example, to CO₂ incubators.

### TECHNICAL FIELD

The present invention relates to an enclosure for processing biological samples, of the type comprising:
a body which itself comprises:
   a structure which internally delimits a space for processing the samples, the processing space opening at the outer side of the structure by way of an opening which is provided in a face of the structure,
   at least one framework element which is fixed in a removable manner to the structure in order to form at least two frames, the frames delimiting, in the opening, at least two zones for access to separate regions of the processing space,
at least two gates which are mounted so as to be movable on the body in order selectively to close and open the access zones.

### BACKGROUND TO THE INVENTION

The invention applies in particular to CO₂ incubators.

Such an incubator comprises means for generating, in the processing space thereof, an atmosphere having a controlled CO₂ content, humidity and temperature. Such incubators are used, for example, in the growth of cells which are present in biological samples taken from patients.

The processing space comprises a plurality of regions delimited by shelves intended to support receptacles containing the biological samples.

In order to limit disturbances produced in the atmosphere of the processing space when it is accessed, such an incubator comprises, in addition to the main door which is generally of metal, gates. These gates which are generally of glass allow selective access to each of the regions of the processing space. In this manner, in order to gain access to a specific region of the processing space, the main door is opened, then the corresponding gate to this region only.

In incubators of the above-mentioned type, the framework element is a metal assembly which comprises all the uprights and all the cross-pieces forming the frames. The glass gates are articulated to this framework element. The framework element is separate and fixed to the structure in a releasable manner. This releasable fixing allows, with the framework element as well as the processing space shelves being removed, the opening of the structure and the work space to be completely cleared in order to clean and decontaminate it.

There is a risk, however, when the framework element is disassembled, of breaking the glass gates which are carried by the framework element. Furthermore, the spatial requirement and the mass of this framework element make the disassembly operation very impractical and further increase the risk of breaking the gates.

Incubators are also known in which the framework element comprises, as in the above case, all the uprights and all the cross-pieces of the frames, but in this case is articulated to the structure. The framework element and the gates articulated thereto then form an auxiliary door.

In order to gain access to a region of the processing space, the main door is opened, then the corresponding gate. The framework element then remains pressed against the structure of the incubator.

In order to carry out the cleaning and decontamination, the main door and the auxiliary door as a whole are opened, which allows total access to be afforded to the opening of the structure.

This structure allows the incubator to be more simply prepared for cleaning and decontaminating the work space thereof, without any risk of breaking the gates. However, the auxiliary door is found to be complex and costly to produce, in particular in order to be able to ensure the leak-tight closure thereof against the structure and to be able to support the mass of the gates.

An object of the invention is to overcome these problems by providing an enclosure of the above-mentioned type whose structure is simple and economical and which limits the risks of damage to the gates when the enclosure is prepared for cleaning and decontamination.

### SUMMARY OF THE INVENTION

To this end, the invention relates to an enclosure of the above-mentioned type, characterised in that the gates are mounted so as to be movable on the structure.

According to specific embodiments, the enclosure can comprise one or more of the following features, taken in isolation or according to all the technically feasible combinations:
the framework element is a cross-piece and at least a first frame is formed by the cross-piece and the structure;
the framework element comprises the two frames;
the enclosure comprises leak-tight means in order to seal in a leak-tight manner the access zones by extending along the peripheries thereof;
the leak-tight means are mounted on the frames;
the leak-tight means which extend along the periphery of the zone delimited by the first frame comprise at least a portion which is mounted in a removable manner on the structure;
that portion is carried by the cross-piece, by means of which it is mounted in a removable manner on the structure;
the structure comprises means for detecting the opening of the gates;
the enclosure further comprises a main door which is articulated to the structure; and
the enclosure constitutes an incubator.

### BRIEF DESCRIPTION OF DRAWINGS

The invention will be better understood from a reading of the description below which is given purely by way of example and with reference to the appended drawings, in which:

FIG. 1 is a schematic front view of a CO₂ incubator according to the invention, the main door being in an open position and the gates in closed positions,

FIG. 2 is a view similar to FIG. 1, the gates being in open positions,

FIG. 3 is a view similar to FIG. 2, the framework elements and the shelves having been withdrawn for cleaning and decontaminating the processing space;

FIG. 4 is an enlarged schematic top view of a framework element of the incubator of FIG. 1;

FIGS. 5 and 6 are partial perspective schematic views showing the connection of the lateral ends of a framework element to the structure of the CO₂ incubator of FIG. 1 and

FIG. 7 is a schematic top view of a framework element according to a variant of the invention.

### DESCRIPTION OF PREFERRED EMBODIMENTS

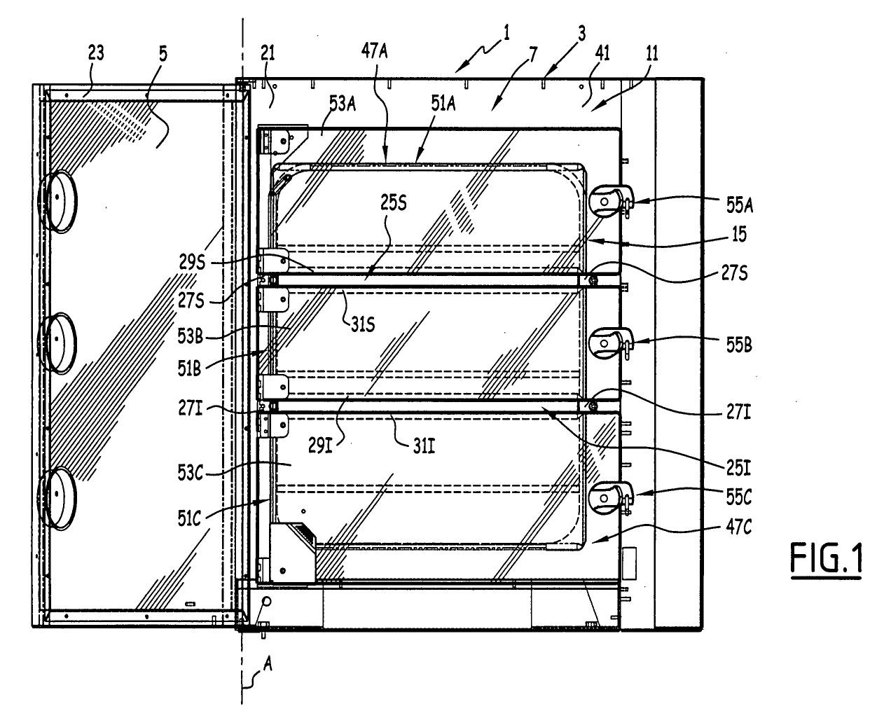

FIG. 1 schematically illustrates a CO₂ incubator 1 which comprises a body 3 and a main door 5.

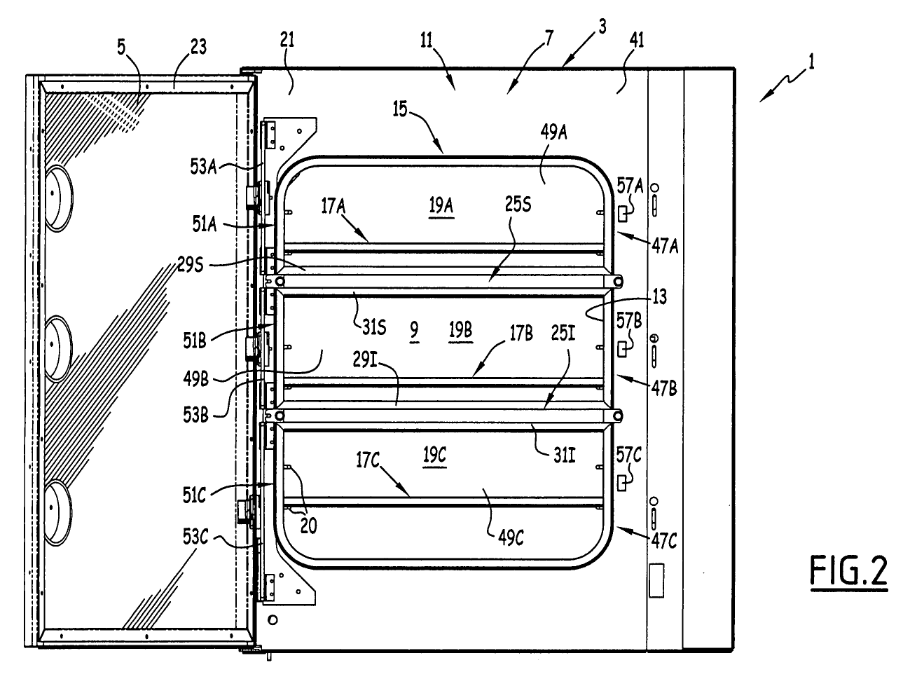

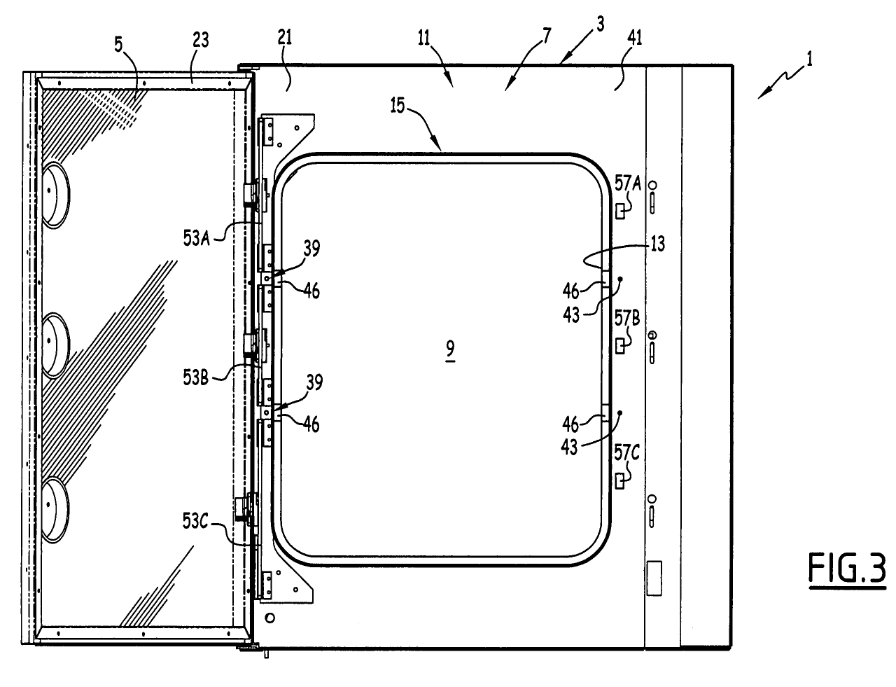

The body 3 comprises a metal structure 7 of generally parallelepipedal form. The structure 7 internally delimits a space 9 for processing biological samples, more particularly visible in FIGS. 2 and 3.

In order to carry out this processing, the incubator 1 conventionally comprises means for generating in the space 9 a controlled atmosphere. In this manner, this atmosphere has a controlled CO₂ content, humidity and temperature.

The processing space 9 opens in the front face 11 of the structure 7 by way of an opening 13 which is more particularly visible in FIGS. 2 and 3. This opening 13 is of generally rectangular form having rounded corners. The front face 11 is provided with a leak-tight seal 15 which extends over the entire periphery of the opening 13.

As can be seen in FIG. 2, the incubator 1 comprises three shelves 17A, 17B and 17C which are arranged one above the other in a removable manner inside the processing space 9.

In this manner, these three shelves 17A, 17B and 17C delimit, in the processing space 9, three separate regions 19A, 19B and 19C which are arranged one above the other.

The shelves 17A, 17B and 17C are, for example, simply positioned on supporting rails 20 (FIG. 2) which are provided on the lateral faces of the processing space 9. The supporting rails 20 are themselves removable.

The door 5 is articulated to the left-hand side 21 of the face 11. In this manner, the door 5 can be moved between a closed position (not illustrated), in which it is pressed against the front face 11, and an open position illustrated by FIGS. 1 to 3, in which the door 5 is remote from the front face 11. In the closed position of the door, a seal 23 provided on the door 5 ensures the leak-tightness between the structure 7 and the door 5. The door 5 then prevents access to the opening 13.

In the open position thereof, the door 5 affords access to this opening 13.

Similarly, the incubator 1 can conventionally comprise magnetic locking means for the door 5 in the closed position thereof.

The body 3 of the incubator 1 comprises, in addition to the structure 7, two framework elements 25S and 25I which are arranged one above the other. These framework elements have a similar structure. The same reference numerals will therefore be used to describe these two elements, followed by the letter I for the lower framework element and the letter S for the upper framework element, respectively.

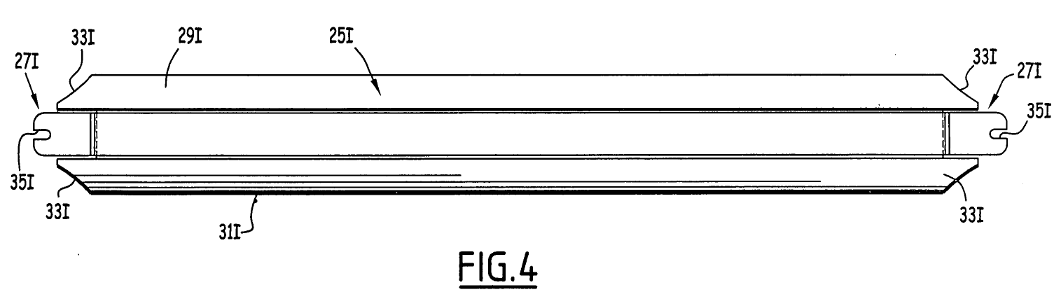

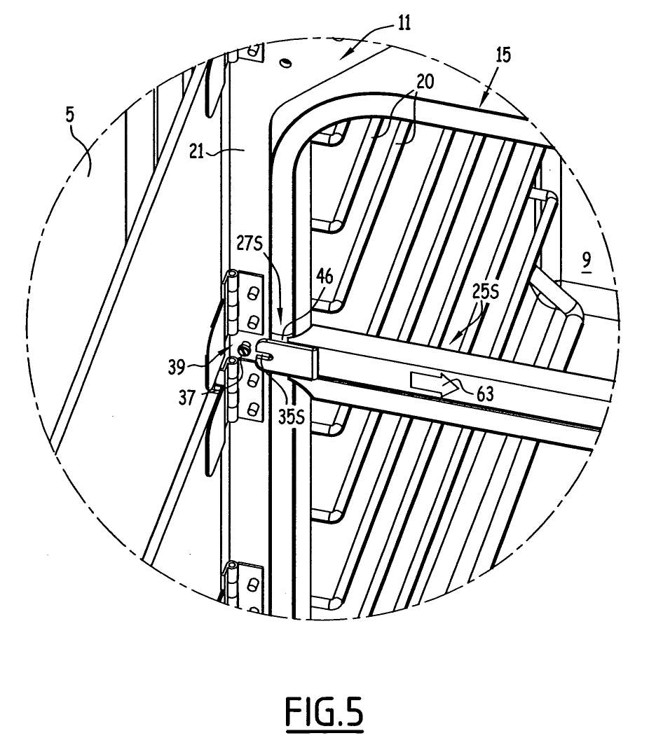

As can be seen in FIG. 4, the framework elements 25I and 25S are metal cross-pieces whose lateral ends 27I, 27S have recesses at the front. In this manner, the central portions of the elements 25I, 25S are recessed relative to the ends 27I, 27S, as can be seen, for example, in FIG. 5. The framework elements 25I, 25S are further provided with rectilinear leak-tight seals 29I, 29S which extend along the upper edges thereof and rectilinear leak-tight seals 31I, 31S which extend along the lower edges thereof. These leak-tight seals comprise end lips 33I, 33S which are chamfered and which converge towards the respective lateral ends 27I, 27S.

The lateral ends 27I, 27S have notches 35I, 35S which allow the framework elements 25I and 25S to be removably fixed to the structure 7.

In greater detail, as is visible in FIG. 5, the left-hand ends 27I, 27S are intended to engage behind collars 37 of two retaining pins 39 which are fixedly joined to the left-hand side 21 of the front face 11. The pins 39 are received in the left-hand notches 35I, 35S and the left-hand ends 27I, 27S are pressed against the structure 7.

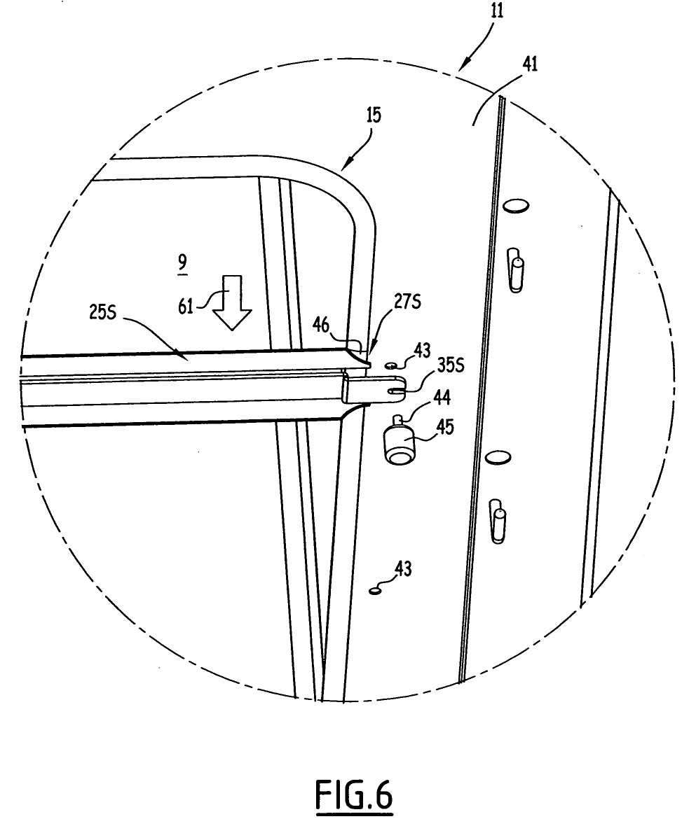

As illustrated by FIG. 6, the right-hand side 41 of the front face 11 of the structure 7 comprises two threaded holes 43 which are intended to receive screw threads 44 which are provided on two knurled knobs 45. The screw threads 44 then engage in the right-hand notches 35I, 35S of the framework elements 25I and 25S.

The knurled knobs 45 press the right-hand ends 27I and 27S of the framework elements 25I, 25S against the front face 11.

It will be appreciated that the seal 15 has recesses 46 which allow the passage of the ends 27I and 27S of the framework elements 15. By way of a variant, these recesses 46 can be replaced with interruptions of the seal 15.

The framework elements 25I and 25S are fixedly joined to the structure 7 and extend transversely through the opening 13. The framework elements 25I and 25S then form, with the structure, three frames 47A, 47B and 47C, as can be seen in FIG. 2.

The lower cross-piece of the upper frame 47A is formed by the framework element 25S and the remainder of the frame 47A, that is to say, the lateral uprights and the upper cross-piece thereof, is formed by the front face 11 of the structure 11.

The horizontal cross-pieces of the intermediate frame 47B are formed by the framework elements 25I and 25S. The two lateral uprights of the frame 47B are formed by the front face 11 of the structure 7.

The upper cross-piece of the lower frame 47C is formed by the framework element 25I. The remainder of this frame 47C is formed by the front face 11 of the structure 7.

As can be seen in FIG. 2, the frames 47A, 47B, 47C delimit, in the opening 13, three zones 49A, 49B and 49C which are arranged one above the other and which respectively allow access to the regions 19A, 19B and 19C of the processing space 9.

The lips 33I and 33S of the seals 29I, 29S, 31I and 31S cover the seal 15 and take the shape thereof. In this manner, the openings 49A, 49B and 49C are surrounded by leak-tight means which are designated 51A, 51B and 51C, respectively.

The leak-tight means 51A comprise an upper portion and two lateral portions which are formed by the leak-tight seal 15. The lower portion of the leak-tight means 51A is formed by the upper seal 29S of the upper framework element 25S.

The leak-tight means 51B comprise two horizontal portions which are formed by the seals 29I and 31S and two lateral portions which are formed by the seal 15.

The leak-tight means 51C comprise an upper portion which is formed by the seal 31I and two lateral portions and a lower portion which is formed by the seal 15.

Owing to the recesses of the lateral ends 27I and 27S of the framework elements 25I and 25S, the portions formed by the seals 15, 29I, 29S, 31I and 31S are located substantially in the same plane. In this manner, all the portions of the leak-tight means 51A, 51B and 51C are located in the same plane, respectively.

As can be seen in FIG. 1, the incubator 1 further comprises three gates 53A, 53B and 53C which are articulated to the left-hand side 21 of the face 11 by hinges in order to be able to be movable relative to the structure 7 independently of each other. The gates 53A, 53B and 53C are, for example, of glass and are more generally transparent in order to be able to discern from outside the contents of the processing space 9.

The gates 53A, 53B and 53C are movable relative to the structure 7 between closed positions, in which they close the zones 49A, 49B and 49C of the opening 13 and are pressed against the leak-tight means 51A, 51B and 51C, and open positions for opening the zones 49A, 49B and 49C, respectively.

The closed positions are illustrated by FIG. 1 and the open positions by FIGS. 2 and 3.

In the closed positions, the various seals 15, 29I, 29S, 31I and 31S are squashed and the leak-tight means 51A, 51B, 51C therefore ensure the leak-tightness between the front face 11 of the structure 7 and the gates 53A, 53B and 53C.

The right-hand sides of the gates 53A, 53B and 53C are provided with means 55A, 55B and 55C for locking the gates in the closed positions thereof.

The structure 7 can further comprise, at the right-hand side 41 of the front face 11 thereof, sensors 57A, 57B and 57C for detecting opening of the gates 53A, 53B and 53C. These can be switches which are electrically connected to the operating and control system of the incubator 1.

In order to gain access to one of the regions 19A, 19B and 19C of the processing space 9 during normal operation of the incubator 1, the door 5, then the corresponding gate 53A, 53B or 53C are opened in succession. The desired operation is then carried out in the selected region of the processing space 9. This can be, for example, the introduction or removal of receptacles containing samples. Only one of the gates 53A, 53B and 53C then being open, the disturbances produced in the atmosphere of the processing space 9 are reduced. At the end of the operation, the selected gate and the door 5 are closed again.

In order to be able to carry out the cleaning and decontamination of the processing space 9, first the door 5 and all the gates 53A, 53B and 53C are opened, as illustrated in FIG. 2.

Next, the framework elements 25I and 25S are withdrawn. This can be brought about by the knobs 45 being unscrewed, then the right-hand ends 27I and 27S of the elements 25I and 25S being moved away from the right-hand side 41 of the front face 11, as indicated by the arrow 61 in FIG. 6, then by the framework elements 25I and 25S being drawn to the right in order to disengage the left-hand ends 27I and 27S thereof from the structure 7, as illustrated by the arrow 63 in FIG. 5. Finally, the shelves 17A, 17B and 17C and the supporting rails 20 are withdrawn.

The incubator 1 is then in the configuration of FIG. 3. The opening 13 and the processing space 9 are completely empty, thereby allowing complete access to the walls of the space 9. Cleaning and decontamination can then be carried out.

In order to use the incubator 1 again, the operations described above are carried out in reverse, in particular with the left-hand ends 27I, 27S of the framework elements 25I and 25S first being engaged behind the collars 37, then the right-hand ends 27I and 27S being fixed by the knobs 45 being screwed in.

In this manner, the framework elements 25I and 25S are light and can be removed very easily for cleaning and decontaminating the incubator 1.

Furthermore, the risk of breaking the gates 53A, 53B and 53C is reduced, because they are mounted on the structure 7 and not on the framework elements, as in the prior art, and therefore are not disassembled when the elements 25I and 25S are removed for cleaning and decontamination. Furthermore, the structure of the framework elements 25I and 25S allows a simple overall structure and reduced costs to be kept for the incubator 1.

It will also be appreciated that the framework elements 25I and 25S allow sensors 57A, 57B and 57C for detecting the opening of the gates 53A, 53B and 53C to be provided, without posing problems in terms of connecting these sensors to the operating and control system of the incubator 1, because these sensors are provided on the structure 7 itself.

The leak-tight means 51A, 51B and 51C can, instead of being constructed by means of fixed portions with respect to the structure 7, comprise at least some removable portions. In this manner, they can be completely removable, continuous elements which are withdrawn for cleaning and decontamination. Similarly, by way of a variant, the leak-tight means 51A to 51C can be provided on the gates themselves rather than on the body 3. It is also possible to envisage not using leak-tight means 51A, 51B and 51C.

In a more general manner, the framework elements can be of a different form associated with a different configuration of the frames formed. In this manner, a separate framework element can be used for each frame, rather than common cross-pieces having a plurality of frames, as in the above example.

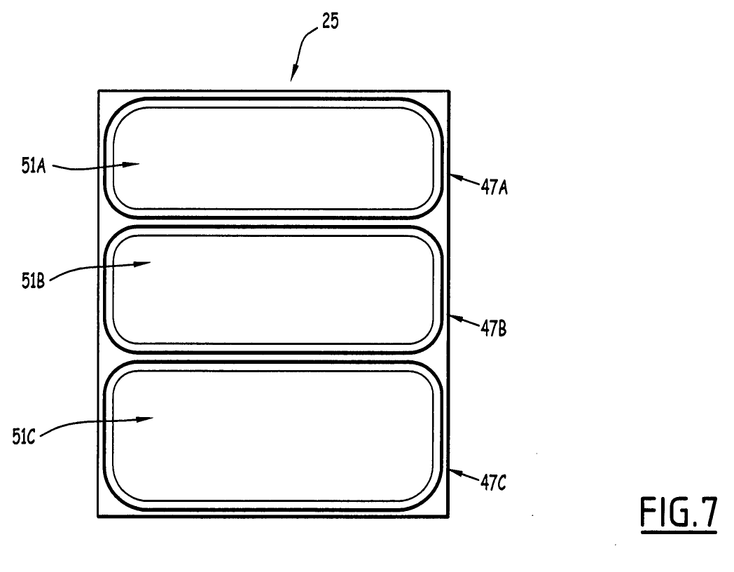

As illustrated by FIG. 7, it is also possible to use a complete framework element 25, that is to say, comprising all the frames 47A, 47B, 47C. However, the gates 53A, 53B, 53C then remain mounted on the structure 7 rather than on that framework element 25 in order to keep a simple structure and to limit the risks of damage to the gates when the incubator 1 is prepared for cleaning and decontamination thereof.

Similarly, in a more general manner, the number of frames 47 formed, and therefore gates 53, can be different from that described above.

The invention can also be applied to any type of enclosure for processing biological samples. Processing is intended to refer to the samples being subjected to a controlled atmosphere, for example, in terms of temperature. Therefore, this term also extends to storage. Thus, the invention can be applied in particular to ovens, refrigerators . . .

1. Enclosure for processing biological samples, of the type comprising:
a body which itself comprises:
a structure which internally delimits a space for processing the samples, the processing space opening at the outer side of the structure by way of an opening which is provided in a face of the structure,
at least one framework element which is fixed in a removable manner to the structure in order to form at least two frames, the frames delimiting, in the opening, at least two zones for access to separate regions of the processing space,
at least two gates which are mounted so as to be movable on the body in order selectively to close and open the access zones,
wherein the gates are mounted so as to be movable on the structure.

2. Enclosure according to claim 1, wherein the framework element is a cross-piece, and in that at least a first frame is formed by the cross-piece and the structure.

3. Enclosure according to claim 1, wherein the framework element comprises the two frames.

4. Enclosure according to claim 1, wherein it comprises leak-tight means in order to seal in a leak-tight manner the access zones by extending along the peripheries thereof.

5. Enclosure according to claim 4, wherein the leak-tight means are mounted on the frames.

6. Enclosure according to claim 2, wherein it comprises leak-tight means in order to seal in a leak-tight manner the access zones by extending along the peripheries thereof.

7. Enclosure according to claim 6, wherein the leak-tight means are mounted on the frames.

8. Enclosure according to claim 7, wherein the leak-tight means which extend along the periphery of the zone delimited by the first frame comprise at least a portion which is mounted in a removable manner on the structure.

9. Enclosure according to claim 8, wherein said portion is carried by the cross-piece, by means of which it is mounted in a removable manner on the structure.

10. Enclosure according to claim 1, wherein the structure comprises means for detecting the opening of the gates.

11. Enclosure according to claim 1 and further comprising a main door which is articulated to the structure.

12. Enclosure according to claim 1 and constituting an incubator.
